(19) United States
(12) Patent Application Publication
(10) Pub. No.: US 2017/0113983 A1
(43) Pub. Date: Apr. 27, 2017

(54) PROCESS FOR THE OXIDATIVE DEHYDROGENATION OF ETHANE

(71) Applicant: SHELL OIL COMPANY, Houston, TX (US)

(72) Inventors: Georgios MITKIDIS, Amsterdam (NL); Maria SAN ROMAN MACIA, Amsterdam (NL); Guus ROSSUM VAN, Amsterdam (NL)

(21) Appl. No.: 15/297,443

(22) Filed: Oct. 19, 2016

(30) Foreign Application Priority Data
Oct. 21, 2015 (EP) 15190865.4

(51) Int. Cl. C07C 5/48

(52) U.S. Cl. CPC C07C 5/48

(57) ABSTRACT

The invention relates to a process for the oxidative dehydrogenation of ethane to ethylene, comprising a reaction step which comprises subjecting a gas stream comprising ethane and air to ethane oxidative dehydrogenation conditions resulting in a gas stream comprising nitrogen, ethane and ethylene; a sorption step which comprises contacting the gas stream comprising nitrogen, ethane and ethylene resulting from the reaction step with a sorption agent which has an affinity for nitrogen which is lower than that for ethane which in turn is lower than that for ethylene, resulting in sorption of ethylene and optionally ethane by the sorption agent and in a gas stream comprising nitrogen and optionally ethane; and a desorption step which comprises desorbing sorbed ethylene and optionally ethane resulting in a gas stream comprising ethylene and optionally ethane.

### FIELD OF THE INVENTION

The present invention relates to a process for the oxidative dehydrogenation of ethane.

### BACKGROUND OF THE INVENTION

It is known to oxidatively dehydrogenate ethane resulting in ethylene, in an oxidative dehydrogenation (oxydehydrogenation; ODH) process. Examples of ethane ODH processes are for example disclosed in U.S. Pat. No. 7,091,377, WO2003064035, US20040147393, WO2010096909 and US20100256432. The oxidative dehydrogenation of ethane converts ethane into ethylene. In this process, a gas stream comprising ethane is contacted with an ODH catalyst and with an oxidant, such as oxygen or air.

In such ODH process, the oxygen is adsorbed on the catalyst's surface. Ethane molecules are then dehydrogenated into ethylene. Usually, the gas stream leaving an ODH process contains a mixture of water, optionally hydrogen, carbon monoxide, carbon dioxide, optionally methane (coming from the ethane feed), ethane and ethylene. In addition, a certain amount of the corresponding carboxylic acid, that is to say acetic acid, may be formed. That is to say, ethylene is initially formed in an ethane ODH process. However, in said same process, said dehydrogenated compound may be further oxidized under the same conditions into the corresponding carboxylic acid. In the case of ethane, the product of said alkane oxidative dehydrogenation process comprises ethylene and optionally acetic acid.

In general, the yield of ethylene (as determined by conversion and selectivity) that is achieved in an ODH process may be relatively low. As a result, a relatively large amount of unconverted ethane leaves the ODH reactor. The proportion of unconverted ethane in the ODH product gas stream may be up to 80 mole % based on the total molar amount of the gas stream. It is desired to recover and then recycle this unconverted ethane.

It is known to separate ethane from ethylene, by means of cryogenic distillation in so-called “C2 splitter” columns. In such cryogenic distillation, a relatively high pressure and a relatively low (cryogenic) temperature are applied to effect the separation of ethane from ethylene. Generally, such “C2 splitter” is preceded by cryogenic distillation wherein light gases and possibly methane (coming from the ethane feed) are first separated from the ethane and ethylene.

An object of the invention is to provide a technically advantageous, efficient and affordable process for the oxidative dehydrogenation of ethane, using air as the oxidant, including a step wherein a product gas stream comprising (unconverted) ethane and ethylene (product) is separated into a gas stream comprising the ethane and another gas stream comprising the ethylene, more especially in a case where such gas stream to be separated comprises a relatively high proportion of unconverted ethane. Such technically advantageous process would preferably result in a lower energy demand and/or lower capital expenditure.

### SUMMARY OF THE INVENTION

Surprisingly it was found that such technically advantageous process, resulting in a lower energy demand and/or lower capital expenditure, may be provided by subjecting a gas stream comprising nitrogen, ethane and ethylene, resulting from subjecting ethane and air to ethane oxidative dehydrogenation conditions, to the following two steps: (1) a sorption step which comprises contacting the gas stream comprising nitrogen, ethane and ethylene with a sorption agent which has an affinity for nitrogen which is lower than that for ethane which in turn is lower than that for ethylene, resulting in sorption of ethylene and optionally ethane by the sorption agent and in a gas stream comprising nitrogen and optionally ethane; and (2) a desorption step which comprises desorbing sorbed ethylene and optionally ethane resulting in a gas stream comprising ethylene and optionally ethane.

Accordingly, the present invention relates to a process for the oxidative dehydrogenation of ethane to ethylene, comprising

a reaction step which comprises subjecting a gas stream comprising ethane and air to ethane oxidative dehydrogenation conditions resulting in a gas stream comprising nitrogen, ethane and ethylene;

a sorption step which comprises contacting the gas stream comprising nitrogen, ethane and ethylene resulting from the reaction step with a sorption agent which has an affinity for nitrogen which is lower than that for ethane which in turn is lower than that for ethylene, resulting in sorption of ethylene and optionally ethane by the sorption agent and in a gas stream comprising nitrogen and optionally ethane; and

a desorption step which comprises desorbing sorbed ethylene and optionally ethane resulting in a gas stream comprising ethylene and optionally ethane.

### BRIEF DESCRIPTION OF THE DRAWINGS

FIG. 1 shows an embodiment of the present invention, in which the gas stream that is subjected to the sorption step additionally comprises components other than nitrogen, ethane and ethylene, namely optionally hydrogen, carbon monoxide and carbon dioxide.

### DETAILED DESCRIPTION OF THE INVENTION

Within the present specification, by “ethane oxidative dehydrogenation catalyst” reference is made to a catalyst for the oxidative dehydrogenation of ethane. Both terms may be used interchangeably. Analogously, by “ethane oxidative dehydrogenation conditions” reference is made to conditions for the oxidative dehydrogenation of ethane, which terms may also be used interchangeably.

Within the present specification, by “substantially no” in relation to the amount of a specific component in a gas stream, it is meant an amount which is at most 1,000, preferably at most 500, preferably at most 100, preferably at most 50, more preferably at most 30, more preferably at most 20, and most preferably at most 10 ppmw of the component in question, based on the amount (i.e. weight) of said gas stream.

Within the present specification, where reference is made to relative (e.g. molar) amounts of components in a gas stream, such relative amounts are to be selected such that the total amount of said gas stream does not exceed 100%.

While the process of the present invention and the streams used in said process are described in terms of “comprising”, “containing” or “including” one or more various described steps and components, respectively, they can also “consist essentially of” or “consist of” said one or more various described steps and components, respectively.”.

In the reaction step of the process of the present invention, a gas stream comprising ethane and air is subjected to ethane oxidative dehydrogenation conditions resulting in a gas stream comprising nitrogen, ethane and ethylene.

In the above-mentioned reaction step, one gas stream comprising ethane and air may be fed to a reactor. Alternatively, two or more gas streams may be fed to the reactor, which gas streams form a combined gas stream comprising ethane and air inside the reactor. For example, one gas stream comprising air and another gas stream comprising ethane may be fed to the reactor separately.

The reactor may be any reactor suitable for the oxidative dehydrogenation of ethane, such as a fixed bed reactor with axial or radial flow and with inter-stage cooling or a fluidized bed reactor equipped with internal and external heat exchangers. It may be a fixed bed multi-tubular reactor, such as a fixed-bed multi-tube shell-and-tube reactor/heat exchanger with catalyst and process flow inside the tubes and a heat transfer fluid (or steam generation) circulated in the shell side, as for example disclosed in US20100256432.

Preferably, subjecting the gas stream comprising ethane and air to ethane oxidative dehydrogenation conditions comprises contacting said gas stream with an ethane oxidative dehydrogenation catalyst, as further described below.

Various processes and reactor set-ups are described in the ODH field and the process of the present invention is not limited in that regard. The person skilled in the art may conveniently employ any of such processes in the reaction step of the process of the present invention.

Suitable oxydehydrogenation processes, including catalysts and other process conditions, include those described in above-mentioned U.S. Pat. No. 7,091,377, WO2003064035, US20040147393, WO2010096909 and US20100256432.

As used herein, the term “reactor feed” is understood to refer to the totality of the gas stream(s) at the inlet(s) of the reactor. Thus, as will be appreciated by one skilled in the art, the reactor feed is often comprised of a combination of one or more gas stream(s), such as an ethane stream, an air stream, a recycle gas stream, etc.

During the oxidative dehydrogenation of ethane, a reactor feed comprising ethane and air is introduced into the reactor, so that a gas stream comprising ethane and air is contacted with an ethane oxidative dehydrogenation catalyst inside that reactor. Optionally, the reactor feed may further comprise minor components of the ethane feed (e.g. methane) or the ethane recycle stream (e.g. ethylene, CO).

Advantageously, in the present invention wherein air is used as the oxidant, there is no need to separately add an inert gas, such as nitrogen, argon or helium, as diluent to the above-mentioned reactor feed comprising ethane and oxygen. For the nitrogen from the air as fed to the reaction step already acts as such diluent. The nitrogen from the air ensures that heat generated during the exothermic reaction is more easily dissipated across the entire reactor volume, which in turn simplies the reactor cooling design. That is, a milder temperature profile is developed which minimizes the number of heat exchangers (for example coolers) required to remove the heat from the reactor.

Further, advantageously, by using air as the oxidant instead of a high purity oxygen gas stream (e.g. at least 95 mole % of oxygen), there is no need to first separate high purity oxygen from air in an expensive air separation unit upstream of the ODH reactor. As will be described herein below, the nitrogen coming from the air is separated in a post-reaction separation procedure which has to be carried out any way in order to remove other components such as above-mentioned hydrogen (H₂) and carbon monoxide (CO).

In the reaction step of the process of the present invention, ethane and air may be added to the reactor as mixed feed, optionally comprising further components therein, at the same reactor inlet. Alternatively, the ethane and air may be added in separate feeds, optionally comprising further components therein, to the reactor at the same reactor inlet or at separate reactor inlets.

In the reaction step of the process of the present invention, the ethane:oxygen molar ratio in the reactor feed may be in the range of from 0.3:1 to 10:1, more preferably 0.7:1 to 5:1. Such ethane:oxygen molar ratios correspond to ethane:air molar ratios of 0.1:1 to 2.1:1 and 0.1:1 to 1.1:1, respectively.

Ethane may be present in the reactor feed in a concentration of at least 5 mole %, more preferably at least 10 mole %, relative to the reactor feed. Further, ethane may be present in the reactor feed in a concentration of at most 70 mole %, more preferably at most 60 mole %, most preferably at most 55 mole %, relative to the reactor feed. Thus, in the present invention, ethane may for example be present in the reactor feed in a concentration in the range of from 5 to 70 mole %, more preferably 10 to 60 mole %, most preferably 10 to 55 mole %, relative to the reactor feed.

In general, the oxygen concentration in the reactor feed should be less than the concentration of oxygen that would form a flammable mixture at either the reactor inlet or the reactor outlet at the prevailing operating conditions.

Air may be present in the reactor feed in a concentration of at least 30 mole %, more preferably at least 40 mole %, most preferably at least 45 mole %, relative to the reactor feed. Further, air may be present in the reactor feed in a concentration of at most 95 mole %, more preferably at most 90 mole %, relative to the reactor feed. Thus, in the present invention, air may for example be present in the reactor feed in a concentration in the range of from 30 to 95 mole %, more preferably 40 to 90 mole %, most preferably 45 to 90 mole %, relative to the reactor feed.

The oxygen concentration in the reactor feed is determined by the above-mentioned relative amount of air, which comprises 21 mole % of oxygen, that is present in the reactor feed. Thus, oxygen may be present in the reactor feed in a concentration of at least 6.3 mole %, more preferably at least 8.4 mole %, most preferably at least 9.5 mole %, relative to the reactor feed. Further, oxygen may be present in the reactor feed in a concentration of at most 20.0 mole %, more preferably at most 18.9 mole %, relative to the reactor feed. Thus, in the present invention, oxygen may for example be present in the reactor feed in a concentration in the range of from 6.3 to 20.0 mole %, more preferably 8.4 to 18.9 mole %, most preferably 9.5 to 18.9 mole %, relative to the reactor feed.

In the above-mentioned reaction step, a reactor feed comprising ethane and air is subjected to ethane oxidative dehydrogenation conditions, which as discussed above, may comprise contacting said gas stream with an ethane oxidative dehydrogenation catalyst so that ethane is converted to ethylene. Suitably, the reactor temperature in said reaction step is in the range of from 100 to 600° C. Preferably, said conversion is effected at a reactor temperature in the range of from 200 to 500° C.

In a preferred embodiment, said conversion of ethane to ethylene is effected at a reactor pressure in the range of from 1 to 50 bar, more preferably 3 to 25 bar, even more preferably 5 to 15 bar.

According to the present invention, the above-mentioned ethane oxidative dehydrogenation catalyst may be any ethane oxidative dehydrogenation catalyst. The amount of such catalyst is not essential. Preferably, a catalytically effective amount of the catalyst is used, that is to say an amount sufficient to promote the ethane oxydehydrogenation reaction.

Further, in the present invention, such catalyst may be a mixed metal oxide catalyst containing molybdenum, vanadium, niobium and optionally tellurium as the metals. Thus, in a preferred embodiment of the present invention, the gas stream comprising ethane and air is contacted with a mixed metal oxide catalyst containing molybdenum, vanadium, niobium and optionally tellurium, resulting in the gas stream comprising nitrogen, ethane and ethylene.

In the present invention, the above-mentioned mixed metal oxide catalyst containing molybdenum, vanadium, niobium and optionally tellurium may have the following formula:

MoₗVₐTe_{b}Nb_{c}Oₙ

wherein:

a, b, c and n represent the ratio of the molar amount of the element in question to the molar amount of molybdenum (Mo);

a (for V) is from 0.01 to 1, preferably 0.05 to 0.60, more preferably 0.10 to 0.40, more preferably 0.20 to 0.35, most preferably 0.25 to 0.30;

b (for Te) is 0 or from >0 to 1, preferably 0.01 to 0.40, more preferably 0.05 to 0.30, more preferably 0.05 to 0.20, most preferably 0.09 to 0.15;

c (for Nb) is from >0 to 1, preferably 0.01 to 0.40, more preferably 0.05 to 0.30, more preferably 0.10 to 0.25, most preferably 0.14 to 0.20; and

n (for O) is a number which is determined by the valency and frequency of elements other than oxygen.

In the present invention, the above-mentioned mixed metal oxide catalyst containing molybdenum, vanadium, niobium and optionally tellurium is a solid, heterogeneous catalyst. Inside a reactor, this heterogeneous catalyst makes up a catalyst bed through which the gas stream comprising air and ethane is sent.

In general, the gas stream comprising nitrogen, ethane and ethylene resulting from the above-described reaction step also comprises water. Water may easily be removed from said gas stream, for example by cooling down the gas stream from the reaction temperature to a lower temperature, for example to a temperature in the range of from 0 to 50° C., suitably 10 to 40° C. or 10 to 30° C., so that the water condenses and can then be removed from the gas stream. In case any carboxylic acid is formed in the present ethane ODH process, such as acetic acid which is the corresponding carboxylic acid originating from ethane, such carboxylic acid would be separated at the same time together with the water. Therefore, preferably, in an embodiment wherein the gas stream resulting from the above-described reaction step comprises nitrogen, ethane, ethylene, water and optionally carboxylic acid, such water and carboxylic acid are removed from such gas stream in the above-mentioned way, preferably before the below-described sorption step is carried out.

Such condensing step, as described above, may be followed by a water wash step in order to remove substantially all carboxylic acid, also preferably before the below-described sorption step is carried out. For example, such water wash may be carried out by contacting the gas stream with water which has an affinity for carboxylic acid.

Further, such condensing step and optional water wash step, as described above, may be followed by a drying step in order to remove substantially all water, also preferably before the below-described sorption step is carried out. For example, such drying may be carried out by contacting the gas stream with an absorption agent which has a high affinity for water, such as for example triethylene glycol (TEG), for example at a temperature in the range of from 30 to 50° C., suitably about 40° C. Alternatively, such drying may be carried out by contacting the gas stream with molecular sieves (or “mol sieves”), suitably at a relatively low temperature in the range of from 10 to 25° C. Using molecular sieves is preferred in a case where the remaining water content should be as low as possible.

The removal of water and any carboxylic acid before the below-described sorption step, as described above, is preferred because then advantageously less sorption agent may be used in the latter step since there is less or substantially no water and carboxylic acid to be sorbed by the sorption agent. Further, by removing water and carboxylic acid at this stage, advantageously less or substantially no water and carboxylic acid will interfere with downstream purification of gas streams coming from the below-described sorption step and/or desorption step.

Further, in the process of the present invention, the gas stream comprising nitrogen, ethane and ethylene resulting from the above-described reaction step is subjected to a sorption step. Suitably, the gas stream that is subjected to the sorption step comprises 40 to 80 mole % of nitrogen, more suitably 50 to 70 mole % of nitrogen; 0 to 40 mole % of ethane, more suitably 0 to 30 mole % of ethane; 0.1 to 35 mole % of ethylene, more suitably 0.5 to 25 mole % of ethylene; and 0 to 20 mole % of oxygen, more suitably 0 to 10 mole % of oxygen. Said relative amounts are based on the total amount of the gas stream.

In the sorption step of the process of the present invention, the gas stream comprising nitrogen, ethane and ethylene resulting from the reaction step is contacted with a sorption agent which has an affinity for nitrogen which is lower than that for ethane which in turn is lower than that for ethylene, resulting in sorption of ethylene and optionally ethane by the sorption agent and in a gas stream comprising nitrogen and optionally ethane. That is to say, the gas stream resulting from the sorption step comprises nitrogen and optionally ethane that is not sorbed by the sorption agent. In particular, the amount of ethane in the gas stream resulting from the sorption step is 0 to 100%, based on the amount of ethane in the gas stream that is subjected to the sorption step. The latter percentage may also be referred to as “ethane rejection” (ethane not being sorbed, but “rejected”). Such “ethane rejection” may be varied by varying the pressure, temperature, nature of the sorption agent and/or configuration of the sorption-desorption system.

Thus, in one embodiment, the sorption step results in sorption of ethylene by the sorption agent and in a gas stream comprising nitrogen and ethane; and the desorption step comprises desorbing sorbed ethylene resulting in a gas stream comprising ethylene.

In another, more preferred embodiment, the sorption step results in sorption of ethylene and ethane by the sorption agent and in a gas stream comprising nitrogen; and the desorption step comprises desorbing sorbed ethylene and ethane resulting in a gas stream comprising ethylene and ethane.

The amount of (rejected) ethane in the gas stream resulting from the sorption step may be at most 100%, or at most 99%, or at most 98%, or at most 95%, or at most 90%, based on the amount of ethane in the gas stream that is subjected to the sorption step. Further, the amount of (rejected) ethane in the gas stream resulting from the sorption step may be at least 0%, or at least 10%, or at least 20%, or at least 30%, or at least 40%, or at least 50%, or at least 60%, based on the amount of ethane in the gas stream that is subjected to the sorption step. Thus, in those embodiments wherein a certain amount of ethane is rejected in the sorption step, the amount of (rejected) ethane in the gas stream resulting from the sorption step may for example be 20 to 100%, or 30 to 100%, or 40 to 100%, or 50 to 100%, or 60 to 100%, based on the amount of ethane in the gas stream that is subjected to the sorption step.

In the sorption step of the process of the present invention, a sorption agent is used. In the present specification, “sorption” means a process in which one substance (the sorption agent) takes up or holds or retains another substance by absorption, adsorption or a combination of both.

Further, said sorption agent used in the sorption step of the process of the present invention has an affinity for nitrogen which is lower than that for ethane which in turn is lower than that for ethylene. This means that under the conditions applied in said sorption step, including pressure and temperature which are further defined herein below, said sorption agent has an affinity for nitrogen which is lower than that for ethane which in turn is lower than that for ethylene. This implies that such sorption agent should be used, that the molar ratio of sorbed ethylene to sorbed ethane is greater than 1:1, assuming equal partial pressures for ethylene and ethane. Preferably, said ratio is equal to or higher than 1.1:1, more preferably equal to or higher than 5:1. Said ratio may be up to 50:1, or may be up to 40:1, or may be up to 30:1, or may be up to 20:1. For example, said ratio is in the range of from 1.1:1 to 50:1 or from 5:1 to 20:1. Sorption agents suitable to be used in the present invention may be selected by comparing the extent of sorption of ethane with the extent of sorption of ethylene, under any given temperature and pressure conditions for a variety of known sorption agents, assuming equal partial pressures for ethylene and ethane. Therefore, a wide range of sorption agents may be used since the only criterion in the present invention is that the sorption agent should have an affinity for nitrogen which is lower than that for ethane which in turn is lower than that for ethylene. Without any limitation, examples of suitable sorption agents are activated carbons; molecular sieves and zeolites (e.g. zeolite 13X, zeolite 5A, ZSM-5, SAPO-34); mesoporous silicas (e.g. SBA-2, SBA-15); porous silicas (e.g. CMK-3, silicate-1); clay heterostructures; Engelhard Titanosilicates (ETS; e.g. ETS-4, ETS-10); porous coordination polymers (PCPs); cation impregnated porous adsorbents and zeolites (e.g. AgA); metal-organic frameworks (MOFs); Zeolitic Imidazolate Framework (ZIFs); and Carbon Organic Frameworks (COFs). Suitable sorption agents are for example disclosed in US20150065767 and US20140249339.

The pressure in the sorption step of the process of the present invention may vary within wide ranges. Preferably, said pressure is higher than atmospheric pressure and at most 30 bar, more preferably at most 15 bar. More preferably, said pressure is of from 5 to 30 bar, more preferably 5 to 15 bar, most preferably 7 to 13 bar. In the reaction step of the process of the present invention, air may be fed at a pressure in the range of from 5 to 30 bar, preferably 5 to 15 bar, more preferably 7 to 13 bar. This implies advantageously that the gas stream comprising nitrogen, ethane and ethylene resulting from the reaction step generally need not be compressed before subjecting it to the sorption step. Thus, preferably, the pressure at which air is fed in the reaction step is the same as the pressure in the sorption step. Still further, the fact that air may be fed in the reaction step at a relatively high pressure makes that the volume of the air is relatively small, which is advantageous in that a smaller ODH reactor may be used, the relative proportion of oxygen in air (78 vol. % of nitrogen and 21 vol. % of oxygen) being relatively small as compared to for example a high purity oxygen gas stream (e.g. at least 95 mole % of oxygen).

The temperature in the sorption step of the process of the present invention may also vary within wide ranges. Preferably, said temperature is in the range of from 0 to 110° C., more preferably 10 to 90° C., most preferably 25 to 80° C. Advantageously, in the present invention, said sorption step may be carried out at a non-cryogenic temperature (e.g. of from 0 to 110° C. as mentioned above).

In the desorption step of the process of the present invention, ethylene and optionally ethane that are sorbed by the sorption agent are desorbed, resulting in a gas stream comprising ethylene and optionally ethane. That is to say, the latter gas stream resulting from the desorption step comprises ethylene and optionally ethane that are desorbed from the sorption agent.

Preferably, in the desorption step of the process of the present invention, desorption is effected by reducing the pressure. That is to say, the pressure in the desorption step is lower than the pressure in the sorption step. This is usually referred to as “Pressure Swing Adsorption” (PSA). In the embodiment wherein desorption in the desorption step is effected by reducing the pressure, the pressure in the sorption step is preferably in the range of from 5 to 30 bar, more preferably 5 to 15 bar, more preferably 7 to 13 bar.

In a case wherein a relatively low pressure (e.g. at most 15 bar) is used in the sorption step, advantageously no or only part of the ethane is sorbed in addition to ethylene. Thus, advantageously, in the sorption step of the process of the present invention, a relatively low pressure is applied (e.g. of from 5 to 15 bar as mentioned above). In addition, such low pressure advantageously results in that relatively less compression of the gas stream may be needed. It is especially advantageous that the pressure that may be needed in the sorption step of the process of the present invention may be the same as the pressure in the preceding (ODH) reaction step. In the latter case, there would be no need at all for any compression of said gas stream in order to carry out said sorption step.

Further, in the embodiment wherein desorption in the desorption step is effected by reducing the pressure, the pressure in the desorption step is preferably in the range of from 0.1 to 3 bar, more preferably 0.5 to 2 bar.

The temperature in the desorption step of the process of the present invention may also vary within wide ranges. Preferably, said temperature is in the range of from 0 to 110° C., more preferably 10 to 90° C., most preferably 25 to 80° C. Advantageously, in the present invention, said desorption step may be carried out at a non-cryogenic temperature (e.g. of from 0 to 110° C. as mentioned above).

Advantageously, the sorption and desorption steps of the process of the present invention make it possible to efficiently separate nitrogen and optionally ethane from a gas stream comprising nitrogen, ethane and ethylene, resulting from the preceding (ODH) reaction step, at a relatively low pressure (e.g. at most 15 bar as mentioned above) and at a non-cryogenic temperature (e.g. of from 0 to 110° C. as mentioned above).

Preferably, the gas stream comprising nitrogen, ethane and ethylene that is subjected to the sorption step of the process of the present invention comprises substantially no water. As described above, preferably, any water is removed from said gas stream before the sorption step is carried out. It is also preferred that said gas stream comprising nitrogen, ethane and ethylene comprises substantially no hydrogen sulfide.

In the embodiment(s), wherein the sorption step of the present process results in sorption of ethylene by the sorption agent and in a gas stream comprising nitrogen and ethane, and the desorption step comprises desorbing sorbed ethylene resulting in a gas stream comprising ethylene, preferably, the present process additionally comprises a distillation step which comprises distilling the gas stream comprising nitrogen and ethane resulting from the sorption step, resulting in a top stream comprising nitrogen and a bottom stream comprising ethane; and optionally a recycle step which comprises recycling the bottom stream comprising ethane resulting from the distillation step to the reaction step.

In the embodiment(s), wherein the sorption step of the present process results in sorption of ethylene and ethane by the sorption agent and in a gas stream comprising nitrogen, and the desorption step comprises desorbing sorbed ethylene and ethane resulting in a gas stream comprising ethylene and ethane, the present process additionally comprises a distillation step which comprises distilling the gas stream comprising ethylene and ethane resulting from the desorption step, resulting in a top stream comprising ethylene and a bottom stream comprising ethane; and optionally a recycle step which comprises recycling the bottom stream comprising ethane resulting from the distillation step to the reaction step.

As is demonstrated in the present Examples, it has surprisingly appeared that advantageously the energy demand, especially the demand for compression and refrigeration energy, is significantly lower as compared to a process wherein a sorption and desorption method is not applied after the ODH reaction step, irrespective of whether in the ODH reaction step of such comparative case only oxygen (no nitrogen) or air is used. Thus, the present process is a process that enables the oxidative dehydrogenation of ethane and subsequent separation of the product stream comprising nitrogen, ethane and ethylene, to recover unconverted ethane and ethylene, in a way that is technically feasible, efficient and affordable since the energy demand is surprisingly lower as compared to the comparative process.

Further, in an embodiment of the process of the present invention, the gas stream comprising nitrogen, ethane and ethylene that is subjected to the sorption step of the process of the present invention additionally comprises components other than said nitrogen, ethane and ethylene, such as carbon monoxide, optionally methane (coming from the ethane feed), optionally hydrogen and carbon dioxide. Therefore, in the present invention, the reaction step may result in a gas stream comprising ethane, ethylene, optionally methane, optionally hydrogen, nitrogen, carbon monoxide and carbon dioxide.

In the above-mentioned embodiment of the process of the present invention, wherein the reaction step results in a gas stream comprising ethane, ethylene, optionally methane, optionally hydrogen, nitrogen, carbon monoxide and carbon dioxide,

the sorption step comprises contacting the gas stream comprising ethane, ethylene, optionally methane, optionally hydrogen, nitrogen, carbon monoxide and carbon dioxide resulting from the reaction step with a sorption agent which has an affinity for hydrogen, nitrogen, carbon monoxide and methane which is lower than that for ethane which in turn is lower than that for carbon dioxide and ethylene, resulting in sorption of carbon dioxide, ethylene and optionally ethane by the sorption agent and in a gas stream comprising optionally hydrogen, nitrogen, carbon monoxide, optionally methane and optionally ethane; and

the desorption step comprises desorbing sorbed carbon dioxide, ethylene and optionally ethane resulting in a gas stream comprising carbon dioxide, ethylene and optionally ethane.

The sorption agents, pressures, temperatures, sorption-desorption method (e.g. PSA) and configuration of the sorption-desorption system as discussed above also apply to the above-mentioned embodiment of the process of the present invention, wherein the gas stream that is subjected to the sorption step (gas stream resulting from the reaction step) comprises ethane, ethylene, optionally methane, optionally hydrogen, nitrogen, carbon monoxide and carbon dioxide.

The sorption step in the above-mentioned embodiment of the process of the present invention may result in a gas stream comprising optionally hydrogen, nitrogen, carbon monoxide, optionally methane and ethane. Preferably, in such case, the process of the present invention additionally comprises a distillation step which comprises distilling the gas stream comprising optionally hydrogen, nitrogen, carbon monoxide, optionally methane and ethane resulting from the sorption step, resulting in a top stream comprising optionally hydrogen, nitrogen, carbon monoxide and optionally methane and a bottom stream comprising ethane. Optionally, said bottom stream comprising ethane is recycled to the reaction step.

Further, preferably, in the above-mentioned embodiment, the process of the present invention additionally comprises a carbon dioxide removal step which comprises removing carbon dioxide from the gas stream comprising carbon dioxide, ethylene and optionally ethane resulting from the desorption step, resulting in a gas stream comprising ethylene and optionally ethane. In said carbon dioxide removal step, carbon dioxide may be removed by any known method, such as treatment with an amine and then with a caustic agent, such as an aqueous monoethanolamine (MEA) absorption system and aqueous NaOH, respectively, as already mentioned above in the introduction of this specification. In a case where said carbon dioxide removal step involves the use of water, said step normally also involves the removal of that water, suitably followed by a drying step. Such drying step may be carried out in order to remove substantially all water and may be carried out in one of the ways as exemplified above in relation to the optional drying step after a condensing step.

Alternatively, said carbon dioxide removal step may be carried out before the sorption step and before any drying step, but after the water condensing step. This is advantageous, first of all in that the gas stream to be subjected to the carbon dioxide removal step may not need to be compressed in a case where the latter gas stream still has a sufficiently high pressure, for example of from 5 to 30 bar or 5 to 15 bar. Secondly, the removal of carbon dioxide before the sorption step is advantageous in that then less sorption agent may be used in the latter step since there is substantially no carbon dioxide to be sorbed by the sorption agent. This alternative embodiment, wherein the carbon dioxide removal step is carried out before the sorption step, may also be applied to cases wherein no air is used as the oxidant, but for example a high purity oxygen gas stream (not according to the invention). In a case where in the present invention, said carbon dioxide removal step is carried out before the sorption step, said carbon dioxide removal step comprises removing carbon dioxide from the gas stream comprising ethane, ethylene, optionally methane, optionally hydrogen, nitrogen, carbon monoxide and carbon dioxide resulting from the reaction step, resulting in a gas stream comprising ethane, ethylene, optionally methane, optionally hydrogen, nitrogen and carbon monoxide.

The above-described embodiments of the process of the present invention, comprising the sorption step and desorption step followed or preceded by the carbon dioxide removal step, may additionally comprise a distillation step wherein either (i) the gas stream resulting from the carbon dioxide removal step as carried out after the desorption step or (ii) the gas stream resulting from the desorption step as carried out after the carbon dioxide removal step and sorption step, respectively, is distilled. Said distillation step will be further described below with reference to said case (i) only.

Preferably, in a case where said gas stream, resulting from said carbon dioxide removal step, comprises ethylene and ethane, said distillation step comprises distilling the gas stream comprising ethylene and ethane resulting from the carbon dioxide removal step, resulting in a top stream comprising ethylene and a bottom stream comprising ethane. Optionally, said bottom stream comprising ethane is recycled to the reaction step.

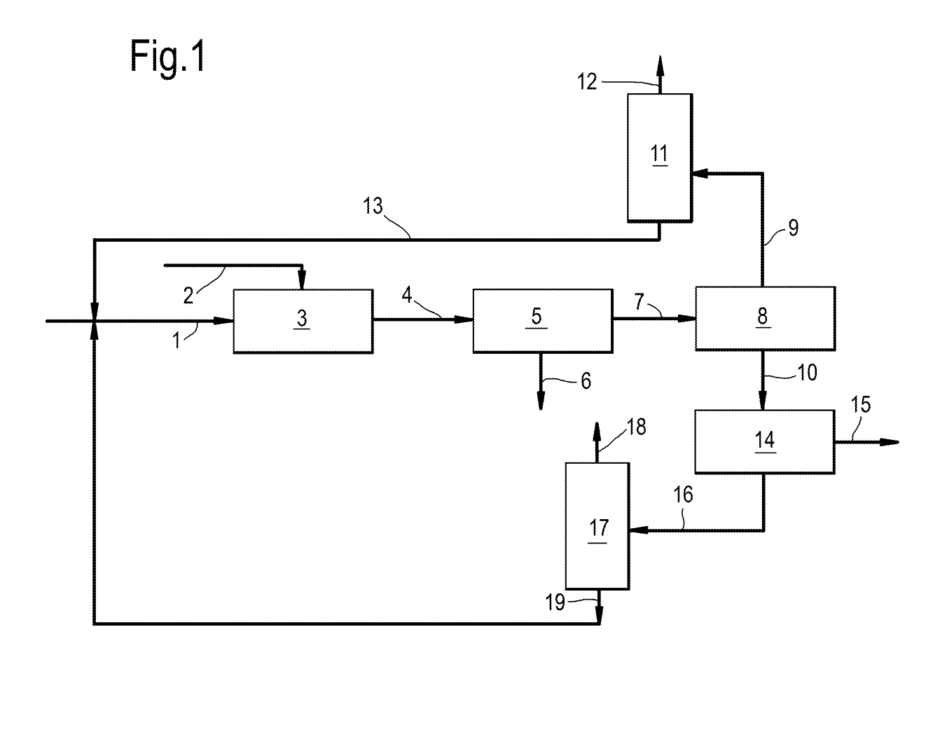

An example of said embodiment of the process of the present invention, wherein the gas stream that is subjected to the sorption step additionally comprises components other than nitrogen, ethane and ethylene, namely optionally hydrogen, carbon monoxide and carbon dioxide, is schematically shown in FIG. 1.

In said FIG. 1, a gas stream 1 comprising ethane and an air stream 2 are fed to an ethane oxidative dehydrogenation (ODH) reactor 3 containing an ODH catalyst and operating under ODH conditions. Product stream 4 originating from ODH reactor 3 comprises water, ethane, ethylene, hydrogen, nitrogen, carbon monoxide and carbon dioxide. Said stream 4 is fed to condensation vessel 5 where water is removed via stream 6. Gas stream 7 comprising ethane, ethylene, hydrogen, nitrogen, carbon monoxide and carbon dioxide originating from condensation vessel 5 is fed to sorption and desorption unit 8. Optionally, before gas stream 7 is fed to sorption and desorption unit 8, it is sent to a drying unit (not shown in FIG. 1) in order to remove substantially all water.

Sorption and desorption unit 8 comprises a sorption agent which has an affinity for hydrogen, nitrogen and carbon monoxide which is lower than that for ethane which in turn is lower than that for carbon dioxide and ethylene. The pressure of gas stream 7 may be of from 5 to 15 bar. Carbon dioxide, part of the ethane and ethylene are sorbed by the sorption agent. Further, a gas stream 9 comprising nitrogen, hydrogen, carbon monoxide and part of the ethane leaves sorption and desorption unit 8, which nitrogen, hydrogen, carbon monoxide and ethane are not sorbed by the sorption agent in sorption and desorption unit 8. Gas stream 9 is sent to distillation column 11.

In distillation column 11, gas stream 9 comprising nitrogen, hydrogen, carbon monoxide and ethane is distilled such that separation between on the one hand nitrogen, hydrogen and carbon monoxide and on the other hand ethane is effected. That is, a top stream 12 comprising nitrogen, hydrogen and carbon monoxide and a bottom stream 13 comprising ethane leave distillation column 11. Said bottom stream 13 is advantageously recycled to ODH reactor 3, for further conversion of the recovered ethane.

After some time, the feed of gas stream 7 to sorption and desorption unit 8 is stopped and the pressure in said unit is reduced. For example, the pressure in sorption and desorption unit 8 may be reduced to a pressure in the range of from 0.1 to 3 bar in a case wherein during the sorption step the pressure is in the range of from 5 to 15 bar, as exemplified above. Through such pressure reduction carbon dioxide, ethane and ethylene that are sorbed by the sorption agent become desorbed. A gas stream 10 comprising carbon dioxide, ethane and ethylene, that are desorbed from the sorption agent, leaves sorption and desorption unit 8 and is sent to carbon dioxide removal unit 14.

Once the desorption is completed, the feed of gas stream 7 to sorption and desorption unit 8 is resumed and the above procedure is repeated.

In carbon dioxide removal unit 14, carbon dioxide is removed, via stream 15, from gas stream 10 comprising carbon dioxide, ethane and ethylene, in a way as exemplified above, that is to say involving the use of water and the removal of that water. A gas stream 16 comprising ethane and ethylene leaves carbon dioxide removal unit 14. Before gas stream 16 is sent to distillation column 17, it is sent to a drying unit (not shown in FIG. 1) in order to remove substantially all water.

As an alternative, carbon dioxide removal unit 14 may be moved to a position in line 7 directly after condensation vessel 5 but before any drying unit in line 7. Further, if there is a drying unit in line 7 (optional), as described above, the drying unit in line 16 may be omitted.

In distillation column 17, gas stream 16 comprising ethane and ethylene is distilled such that separation between on the one hand ethylene and and on the other hand ethane is effected. That is, a top stream 18 comprising ethylene and a bottom stream 19 comprising ethane leave distillation column 17. Said bottom stream 19 is advantageously recycled to ODH reactor 3, for further conversion of the recovered ethane.

If in the setup of FIG. 1, a gas stream 1 comprising ethane of sufficiently high pressure (for example in the range of from 5 to 15 bar) is fed to ODH reactor 3, gas compressors would advantageously only be needed in line 2 (compression of air) and in line 10 (compression of gas stream 10 leaving sorption and desorption unit 8, after desorption, and entering carbon dioxide removal unit 14).

The invention is further illustrated by the following Examples.

### Examples A-C and Comparative Examples D-E

In Examples A, B and C exemplifying the present invention and in Comparative Example E, a gas stream comprising ethane having a temperature of 40° C. and a pressure of 10 bar is fed to an ethane oxidative dehydrogenatation (ODH) reactor. In addition, a gas stream comprising air having a temperature of 40° C. and being compressed to 10 bar by a compressor comprising 3 compression stages, is fed to the ODH reactor. Said 2 gas streams form a combined gas stream comprising ethane and air inside the ODH reactor, which combined gas stream comprises 30 mole % of ethane and 70 mole % of air (ethane:air molar ratio=0.4), that is to say 30 mole % of ethane, 15 mole % of oxygen and 55 mole % of nitrogen (ethane:oxygen molar ratio=2). The ODH reactor contains an ethane oxidative dehydrogenatation (ODH) catalyst and is operated under ODH conditions, including a temperature in the range of from 200 to 500° C. and a pressure of 10 bar. The conversion of ethane is 80% and the selectivity to ethylene is 100%.

In Examples A, B and C and in Comparative Example E, a product stream comprising 5 mole % of ethane, 3 mole % of oxygen, 21 mole % of ethylene, 21 mole % of water and 50 mole % of nitrogen leaves the ODH reactor. Said product stream is cooled to a temperature of 40° C., thereby condensing out the water which is then separated. Any remaining water in said product stream is removed in a drying unit. After said water removal, said product stream is a gas stream comprising 6.3 mole % of ethane, 3.8 mole % of oxygen, 26.6 mole % of ethylene and 63.3 mole % of nitrogen. In Examples A, B and C (not in Comparative Example E), the latter gas stream is fed to a sorption and desorption unit, which comprises a sorption agent which has an affinity for nitrogen (and for oxygen) which is lower than that for ethane which in turn is lower than that for ethylene. All of the ethylene is sorbed by the sorption agent. In Example A, all of the ethane is also sorbed by the sorption agent (ethane rejection=0%). In Example B, 50% of the ethane is also sorbed by the sorption agent (ethane rejection=50%). In Example C, no ethane is sorbed by the sorption agent (ethane rejection=100%).

In Example A, a gas stream comprising nitrogen and oxygen, and having a temperature of 40° C. and a pressure of 10 bar, leaves the sorption and desorption unit, which nitrogen and oxygen are not sorbed by the sorption agent in the sorption and desorption unit.

In Examples B and C, a gas stream comprising nitrogen, oxygen and ethane, and having a temperature of 40° C. and a pressure of 10 bar, leaves the sorption and desorption unit, which nitrogen, oxygen and ethane are not sorbed by the sorption agent in the sorption and desorption unit. The latter gas stream is compressed to 18 bar by a compressor comprising 1 compression stage and then cooled to a temperature of −146° C. (Example B) or −134° C. (Example C) in two parallel heat exchangers utilizing the low temperature of the top and bottom streams coming from below-described distillation column A Then said stream is fed to a distillation column having 4 theoretical stages, hereinafter referred to as distillation column A, and distilled, resulting in a top stream comprising nitrogen and oxygen and having a temperature of −159° C. and a pressure of 17.5 bar and in a bottom stream comprising ethane and having a temperature of −31° C. (Example B) or −21° C. (Example C) and a pressure of 18 bar. Said top and bottom streams are used to cool the feed streams in order to minimize condenser duty in distillation column A, which is provided by a cascaded methane-ethylene-propane refrigeration cycle.

After some time, in Examples A, B and C, the feed of the gas stream to the sorption and desorption unit is stopped and the pressure in said unit is reduced from 10 bar to 1 bar, thereby inducing the desorption step of the process of the present invention. The sorbed components (ethylene and optionally ethane) subsequently become desorbed from the sorption agent and leave the sorption and desorption unit at a temperature of 40° C. and a pressure of 1 bar. In all of Examples A, B and C, the latter gas stream is advantageously enriched in ethylene as compared to the gas stream that is fed to the sorption and desorption unit: in Example A, the gas stream leaving the sorption and desorption unit upon desorption comprises all of the ethylene and all of the ethane; in Example B, said gas stream comprises all of the ethylene and 50% of the ethane; and in Example C, said gas stream comprises all of the ethylene and no ethane.

In Examples A and B, the gas stream leaving the sorption and desorption unit upon desorption and comprising ethylene and ethane is compressed to 16.5 bar by a compressor comprising 3 compression stages and then cooled to a temperature of −32° C. (Example A) or −34° C. (Example B) in two parallel heat exchangers utilizing the low temperature of the top and bottom streams coming from below-described distillation column B Then said stream is fed to a distillation column having 99 theoretical stages, hereinafter referred to as distillation column B, and distilled, resulting in a top stream comprising ethylene and having a temperature of −38° C. and a pressure of 15.5 bar and in a bottom stream comprising ethane and having a temperature of −16° C. and a pressure of 16 bar. Said top and bottom streams are used to cool the feed streams in order to minimize condenser duty in distillation column B, which is provided by a propane refrigeration cycle.

In Table 1 below, the reflux ratios and the distillate-to-feed ratios needed to achieve the above separations in distillation columns A and B in Examples A, B and C are mentioned. By said “reflux ratio”, reference is made to the molar ratio of the molar flow rate of the “reflux stream”, which is that part of the stream that leaves the condenser at the top of the distillation column which is sent back to that column, divided by the molar flow rate of the “distillate”, which is that part of the stream that leaves the condenser at the top of the distillation column which is not sent back to that column By said “distillate-to-feed ratio”, reference is made to the molar ratio of the molar flow rate of said “distillate” divided by the molar flow rate of the feed stream that is fed to that column (the “feed”).

TABLE 1

| | Distillation | Reflux | Distillate-to- |
|---|---|---|---|
| Example | column | ratio | feed ratio |
| A | B | 2.5 | 0.79 |
| B | A | 0.2 | 0.95 |
| B | B | 2.1 | 0.88 |
| C | A | 0.3 | 0.90 |

In Comparative Example D, a gas stream comprising air of ambient temperature and pressure is fed to an air separation unit (ASU). The ASU is operated such that the following 2 streams leave the ASU: 1) a gas stream comprising nitrogen having a temperature of 40° C. and a pressure of 20 bar (which nitrogen can be subsequently stored); and 2) a gas stream comprising oxygen (purity of 99.5 mole %; 0.5 mole % of nitrogen) having a temperature of 40° C. and a pressure of 10 bar. Said gas stream comprising oxygen is fed to an ethane oxidative dehydrogenatation (ODH) reactor. In addition, a gas stream comprising ethane having a temperature of 40° C. and a pressure of 10 bar is fed to an ethane oxidative dehydrogenatation (ODH) reactor. Said 2 gas streams form a combined gas stream comprising ethane and oxygen (and a minor amount of nitrogen) inside the ODH reactor, which combined gas stream comprises 67 mole % of ethane and 33 mole % of oxygen (ethane:oxygen molar ratio=2). The ODH reactor contains an ethane oxidative dehydrogenatation (ODH) catalyst and is operated under ODH conditions, including a temperature in the range of from 200 to 500° C. and a pressure of 10 bar. The conversion of ethane is 80% and the selectivity to ethylene is 100%.

In Comparative Example D, a product stream comprising 10.5 mole % of ethane, 5.3 mole % of oxygen, 42.1 mole % of ethylene and 42.1 mole % of water (and a minor amount of nitrogen) leaves the ODH reactor. Said product stream is cooled to a temperature of 40° C., thereby condensing out the water which is then separated. Any remaining water in said product stream is removed in a drying unit. After said water removal, said product stream is a gas stream comprising 18.2 mole % of ethane, 9.2 mole % of oxygen and 72.6 mole % of ethylene (and a minor amount of nitrogen). The latter gas stream is compressed to 18 bar by a compressor comprising 1 compression stage and then cooled to a temperature of −34° C. Then said stream is fed to a distillation column having 10 theoretical stages, hereinafter referred to as distillation column C, and distilled, resulting in a top stream comprising oxygen (and a minor amount of nitrogen) and having a temperature of −130° C. and a pressure of 17.5 bar and in a bottom stream comprising ethane and ethylene and having a temperature of −30° C. and a pressure of 18 bar. Said top stream is used to cool the feed streams in order to minimize condenser duty in distillation column C, which is provided by a cascaded methane-ethylene-propane refrigeration cycle.

In Comparative Example D, said bottom stream comprising ethane and ethylene is expanded to 16 bar and a temperature of −33° C. Then said stream is fed to a distillation column having 99 theoretical stages, hereinafter referred to as distillation column D, and distilled, resulting in a top stream comprising ethylene and having a temperature of −38° C. and a pressure of 15.5 bar and in a bottom stream comprising ethane and having a temperature of −16° C. and a pressure of 16.2 bar. Said top and bottom streams are used to cool the feed streams in order to minimize condenser duty in distillation column C.

In Table 2 below, the reflux ratios and the distillate-to-feed ratios needed to achieve the above separations in distillation columns C and D in Comparative Example D are mentioned.

TABLE 2

| Comparative | Distillation | Reflux | Distillate-to- |
|---|---|---|---|
| Example | column | ratio | feed ratio |
| D | C | 9.2 | 0.10 |
| D | D | 2.3 | 0.79 |

In Comparative Example E, the gas stream comprising ethane, oxygen, ethylene and nitrogen resulting from the ODH reaction and subsequent water removal, is compressed to 18 bar by a compressor comprising 1 compression stage and then cooled to a temperature of −79° C. Then said stream is fed to a distillation column having 5 theoretical stages, hereinafter referred to as distillation column E, and distilled, resulting in a top stream comprising nitrogen and oxygen and having a temperature of −158° C. and a pressure of 17.5 bar and in a bottom stream comprising ethane and ethylene and having a temperature of −30° C. and a pressure of 17.6 bar. Said top stream is used to cool the feed streams in order to minimize condenser duty in distillation column E, which is provided by a cascaded methane-ethylene-propane refrigeration cycle.

In Comparative Example E, said bottom stream comprising ethane and ethylene is expanded to 16 bar and a temperature of −34° C. Then said stream is fed to a distillation column having 99 theoretical stages, hereinafter referred to as distillation column F, and distilled, resulting in a top stream comprising ethylene and having a temperature of −38° C. and a pressure of 15.5 bar and in a bottom stream comprising ethane and having a temperature of −16° C. and a pressure of 16.2 bar. Said top and bottom streams are used to cool the feed streams in order to minimize condenser duty in distillation column E.

In Table 3 below, the reflux ratios and the distillate-to-feed ratios needed to achieve the above separations in distillation columns E and F in Comparative Example E are mentioned.

TABLE 3

| Comparative | Distillation | Reflux | Distillate-to- |
|---|---|---|---|
| Example | column | ratio | feed ratio |
| E | E | 1.6 | 0.66 |
| E | F | 2.4 | 0.79 |

In all of the (Comparative) Examples, ethane containing streams separated in the distillation columns may be recycled to the ODH reactor at 10 bar. The temperature reduction by reducing the pressure of such recycle ethane containing streams to 10 bar, as well as the temperature reduction by reducing the pressure of nitrogen and oxygen containing top (vent) streams to atmospheric pressure, are utilized to cool the feed streams to the distillation columns and in this way the condenser duty provided by refrigeration is reduced.

In Table 4 below, the compression and refrigeration energy needed to convert ethane into ethylene and to separately recover ethane and ethylene from the product stream is included for all of Examples A-C and Comparative Examples D-E. Said energy is expressed as kilowatt hour (“kWh”; 1 kWh=3.6 megajoules) per kilogram (kg) of ethylene.

TABLE 4

| | | kWh/kg |
|---|---|---|
| | | of |
| Ex. | Configuration | ethylene |
| A | air [O₂ + N₂] | 0.52 |
| | PSA [100% N₂ + O₂ rejection + 0% ethane rejection] | |
| | 1 distillation step separating desorbed ethylene and ethane | |
| B | air [O₂ + N₂] | 0.58 |
| | PSA [100% N₂ + O₂ rejection + 50% ethane rejection] | |
| | 1 distillation step separating desorbed ethylene and ethane | |
| | 1 distillation step separating rejected N₂ + O₂ and ethane | |
| C | air [O₂ + N₂] | 0.46 |
| | PSA [100% N₂ + O₂ rejection + 100% ethane rejection] | |
| | 1 distillation step separating rejected N₂ + O₂ and ethane | |
| D* | O₂ [no N₂] + distillation only [no PSA] | 0.97 |
| E* | air [O₂ + N₂] + distillation only [no PSA] | 0.93 |

*= comparative

From Table 4 above, it surprisingly appears that the energy needed to convert ethane into ethylene and to separately recover ethane and ethylene from the product stream is advantageously lowest in case the process of the present invention is carried out. That is, in all of Examples A, B and C, which exemplify the process of the present invention wherein in the ODH reaction step air is used and in the subsequent product separation step a sorption and desorption method (in said Examples: PSA method) is applied, said energy is advantageously lower than the energy needed to effect the same in those cases wherein a sorption and desorption method is not applied after the ODH reaction step, but only distillation steps are performed (as in Comparative Examples D and E), both when only oxygen (no nitrogen) is used in the ODH reaction step (Comparative Example D) and when air is used in the ODH reaction step (Comparative Example E).

Thus, surprisingly, this advantageous different energy effect obtained with the process of the present invention, as compared to the processes wherein only distillation steps are performed, is even obtained in cases where said sorption and desorption step is followed by 1 distillation step (Examples A and C) or 2 distillation steps (Example B) to recover the ethane and ethylene.

What is claimed is:

1. A process for the oxidative dehydrogenation of ethane to ethylene, comprising
a reaction step which comprises subjecting a gas stream comprising ethane and air to ethane oxidative dehydrogenation conditions resulting in a gas stream comprising nitrogen, ethane and ethylene;
a sorption step which comprises contacting the gas stream comprising nitrogen, ethane and ethylene resulting from the reaction step with a sorption agent which has an affinity for nitrogen which is lower than that for ethane which in turn is lower than that for ethylene, resulting in sorption of ethylene and ethane by the sorption agent and in a gas stream comprising nitrogen and optionally ethane; and
a desorption step which comprises desorbing sorbed ethylene and ethane resulting in a gas stream comprising ethylene and ethane.

2. The process according to claim 1, wherein desorption in the desorption step is effected by reducing the pressure.

3. The process according to claim 2, wherein the pressure in the sorption step is in the range of from 5 to 30 bar, and the pressure in the desorption step is in the range of from 0.1 to 3 bar.

4. The process according to claim 3, wherein in the reaction step air is fed at a pressure in the range of from 5 to 15 bar.

5. The process according to claim 1, wherein the sorption step results in sorption of ethylene and ethane by the sorption agent and in a gas stream comprising nitrogen; and the desorption step comprises desorbing sorbed ethylene and ethane resulting in a gas stream comprising ethylene and ethane.

6. The process according to claim 5, additionally comprising
a distillation step which comprises distilling the gas stream comprising ethylene and ethane resulting from the desorption step, resulting in a top stream comprising ethylene and a bottom stream comprising ethane; and
a recycle step which comprises recycling the bottom stream comprising ethane resulting from the distillation step to the reaction step.
